(19) United States
(12) Patent Application Publication
(10) Pub. No.: US 2023/0414406 A1
(43) Pub. Date: Dec. 28, 2023

(54) Freeze Frame Inserts

(71) Applicant: Roy Edwards Romano, Alvin, TX (US)

(72) Inventor: Roy Edwards Romano, Alvin, TX (US)

(21) Appl. No.: 18/334,990

(22) Filed: Jun. 14, 2023

Related U.S. Application Data
(60) Provisional application No. 63/352,132, filed on Jun. 14, 2022.

(51) Int. Cl. A61F 7/02; G02C 11/00

(52) U.S. Cl. CPC A61F 7/02; A61F 2007/0233; G02C 11/00

(57) ABSTRACT

Freeze frame insert is a cooling device that is intended to provide users with a cooling surface for eyewear frames. To accomplish this, the device includes insulating sleeves and gel packets. As the insulating sleeves and gel packets can be made of varying dimensions, this allows the device to be used with any size or shape of glasses or eyewear. As such, the user can choose any style of frame or design without needing to specifically consider accommodating an integrated chilling method ahead of time. Further, both the insulating sleeve and the cooling gel packet can be resized as necessary during manufacture to fit any frame, regardless of the size of the shape of the eyewear. The insulating sleeve may furthermore be made from a more flexible material, allowing one insulating sleeve to flex to fit a variety of frames.

### FIELD OF THE INVENTION

This invention relates generally to cooling add-ons for use with eyewear. More particularly, the present invention relates to cooling gel packets that are coupled with an insulating sleeve that may be used with a variety of eyewear already in existence.

### BACKGROUND OF THE INVENTION

Users of eyewear may often wish to chill the frames of the eyewear to create a cooling effect on the sides of the heads and temples. Such a cooling effect is often desirable to lower body temperature in hot environments or simply to create a pleasant sensation from the feeling of the cool material against warm skin. Currently, the main cooling means for most eyewear involves having the cooling means or material integrated directly into the eyewear frame. However, such integration with the frame poses a two-fold problem.

Firstly, the insulation for the cooling material can only be as good as the frames of the eyewear allow. This requires the eyewear frames be either made with or integrated with a separate insulating material, which restricts the construction and design of the eyewear.

Secondly, the eyewear itself must be chilled to be of use. This often means that the user must remove the eyewear in order to chill it. This creates a situation in which the user is only able to keep using the eyewear for a fraction of the time, and the eyewear must be continuously removed to chill it again as the material warms up through use. Furthermore, the resulting temperature differential in the lenses can lead to fogging, creating obstructions in vision for the user.

An objective of the present invention is to provide a removable, interchangeable means of cooling that works with a wide variety of eyewear. By using insulating sleeves of varying sizes combined with individual cooling gel packets, the need to remove the entire eyewear for cooling is obviated. As soon as the eyewear begins to warm up, the user may simply obtain another cooling gel packet that is already cold and swap it with the now-warm gel packet. Rather than needing to remove the entire eyewear for a period of time to chill it, the user can refresh the chilling effect immediately while retaining use of the eyewear. This has the effect of increasing the available use-time of the eyewear and allows for perpetual cooling of the eyewear through swapping of the cooling gel packets.

An objective of the present invention is to provide users with a cooling aid for eyewear frames, that comprises insulating sleeves and gel packets. As the insulating sleeves and gel packets can be made of varying dimensions, this allows the invention to be used with any size or shape of glasses or eyewear. As such, the user can choose any style of frame or design without needing to specifically consider accommodating an integrated chilling method ahead of time. The present invention ensures complete versatility for use with any existing eyewear on the market, allowing users to purchase eyewear freely without the need for retrofitting the eyewear with a cooling method.

### SUMMARY OF THE INVENTION

Freeze frame insert is intended to provide users with a cooling aid for eyewear frames that comprises insulating sleeves and gel packets. As the insulating sleeves and gel packets can be made of varying dimensions, this allows the invention to be used with any size or shape of glasses or eyewear. As such, the user can choose any style of frame or design without needing to specifically consider accommodating an integrated chilling method ahead of time. Both the insulating sleeve and the cooling gel packet can be resized as necessary during manufacture to fit any frame, regardless of the size or shape of the eyewear. The insulating sleeve may furthermore be made from a more flexible material, allowing one insulating sleeve to flex to fit a variety of frames. This ensures the invention has complete versatility for use with any existing eyewear on the market, allowing users to purchase eyewear freely without the need for retrofitting the eyewear with a cooling method.

### BRIEF DESCRIPTION OF THE DRAWINGS

FIG. 1 is a top-front-left perspective view of the present invention, wherein an insulating sleeve is represented by darker solid lines, a gel packet is represented by broken lines, and an eyewear frame is represented by thinner sold lines.

FIG. 2 is a top-front-left perspective view of the present invention, wherein the gel packet is shown protruding outside the insulating sleeve.

FIG. 3 is a perspective side view of the gel packet.

FIG. 4 is a sectional view of the gel packet showing the gel on the interior.

FIG. 5 is a front view of the insulating sleeve wherein the gel packet is shown within the storage cavity.

FIG. 6 is a rear side view of the insulating sleeve.

FIG. 7 is a side perspective view of the insulating sleeve.

FIG. 8 is a perspective view of the eyewear frame.

### DETAIL DESCRIPTIONS OF THE INVENTION

All illustrations of the drawings are for the purpose of describing selected versions of the present invention and are not intended to limit the scope of the present invention.

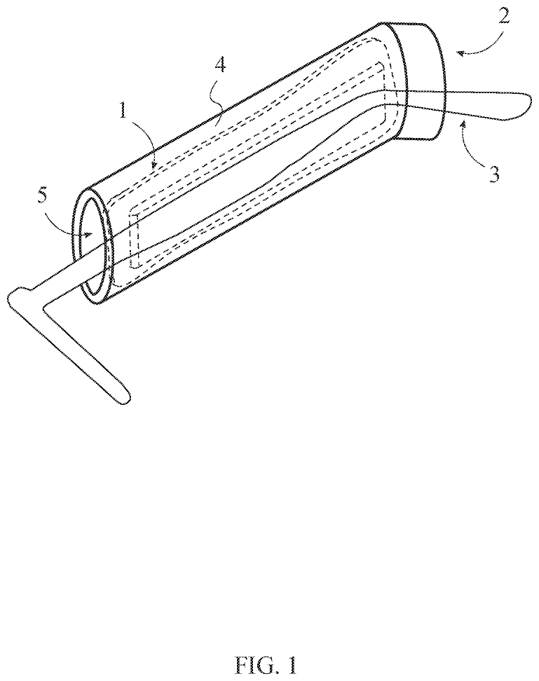

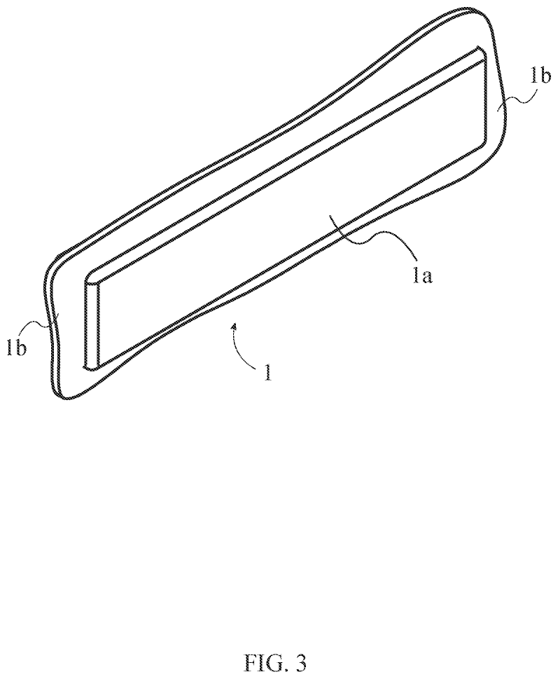

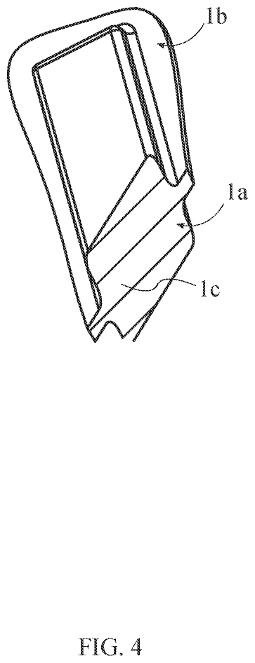

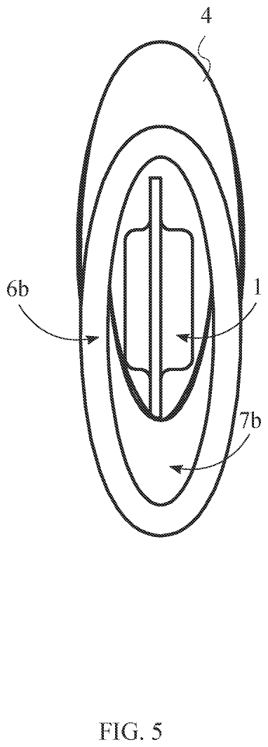

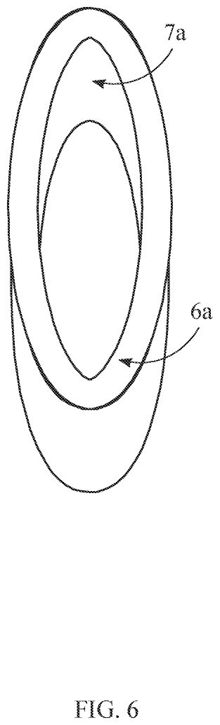

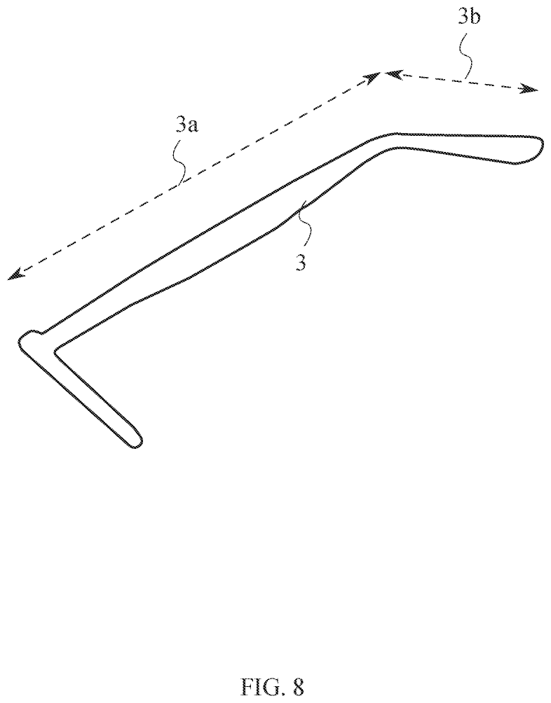

The following description is in reference to FIG. 1 through FIG. 8. According to a preferred embodiment, the present invention comprises a gel packet 1, an insulating sleeve 2, and an eyewear frame leg 3. In the ideal embodiment, the gel packet 1 further comprises an exterior that is made of pliable material such as plastic or polyurethane but may be made of more rigid material in other embodiments. The gel packet 1 is constructed in such a way that the gel packet 1 has an exterior and a hollow interior. More specifically, the gel packet 1 comprises a central cavity 1a, and a plurality of sealed edges 1b. The central cavity 1a is partially filled with gel 1c, and the sealed edges 1b are perimetrically positioned around the central cavity 1a. In other words, the hollow interior is filled with a gel or gel-like substance for the purpose of retaining cold when placed into an external cold source. The exterior of the gel packet 1 is firmly sealed throughout, so as to avoid any discharge or escape from the gel on the interior. An example of such a material includes a super-absorbent polymer, but any material being both malleable and allowing for heat retention and transfer may be used inside the gel packet 1. The gel packet 1 may be sealed by any means that creates a firm seal to retain the gel. One example of such means includes but is not limited to heat sealing plastic or plastic aluminum sheeting to form the sealed gel packet, though any similar processes or materials that achieve the same effect should be considered within the spirit and scope of this invention. The gel packet 1 may be sized at manufacture to fit a variety of different eyewear or fit in a variety of different sizes of insulating sleeves.

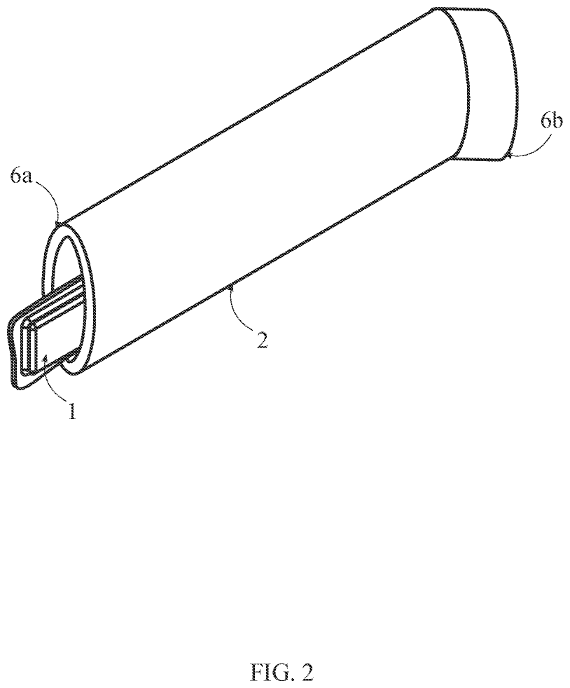

According to the preferred embodiment, the insulating sleeve 2 comprises a sleeve body 4, a storage cavity 5, a first end 6a, and a second end 6b. As seen in FIG. 2, the first end 6a is positioned opposite the second end 6b across the sleeve body 4. Preferably, the storage cavity 5 is positioned within the sleeve body 4 and the storage cavity 5 traverses the sleeve body 4 through the first end 6a and the second end 6b. Accordingly, the storage cavity 5 creates a first hole 7a and a second hole 7b adjacent to the first end 6a and the second end 6b respectively. The sleeve body 4 and both the first hole 7a and second hole 7b may come in a variety of sizes and shapes to accommodate a variety of eyewear. In other words, the storage cavity 5 is positioned within the insulating sleeve 2, in order to house the gel packet 1 and the eyewear frame leg 3 while using the present invention.

In the ideal embodiment as shown in the figures, the insulating sleeve 2 has the shape of a roughly elliptical tube that should be sized to accept most standard sizes of legs or frames from glasses or other eyewear. The sleeve body 4 may be formed from a single piece of material or may be formed from multiple pieces of material stitched together. In some embodiments, the first hole 7a or second hole 7b may be sealed off to increase the insulative properties of the insulating sleeve 2 and to help hold the gel packet 1 in place, creating only one entry point or exit point for both the gel pack 1 and heat to escape. In other words, at least one of the first end 6a and the second end 6b may be sealable. It is preferred that the insulating sleeve 2 should serve to provide a receptacle for the gel packet 1 and to help keep the gel packet 1 cold during use. In the preferred embodiment, the sleeve body 4 is made from a material that is both flexible and has insulating properties, such as neoprene or spandex, though any material having similar properties is ideal. The insulating properties of the insulating sleeve 2 are intended to help prevent the gel from warming up during use, thus extending the time the gel packet 1 can be used before needing to be chilled again. However, other embodiments of the sleeve body 4 may be made from other materials that are either rigid or lack insulating properties.

According to the preferred embodiment, the gel packet 1 is removably positioned within the storage cavity 5 in such a way that the insulating sleeve 2 conceals the gel packet 1 within the storage cavity 5. These insulated gel packets will provide users with a freely detachable means to cool their forehead and temples without needing to remove the eyewear to chill the eyewear itself. Further, when in use, the eyewear frame leg 3 is removably threaded through the insulating sleeve 2. The elastic nature of the sleeve body 4 enables to encapsulate the gel packet 1 and the eyewear frame leg 3 in a snug fashion and thereby help with providing the necessary coldness to the frames quickly. To that end, when in use, the gel packet 1 within the insulating sleeve 2 is pressed against the eyewear frame leg 3. In order to provide the necessary cooling effect, the gel packet 1 is pre-cooled before being positioned within the insulating sleeve 2. Thus, the insulating sleeve 2 provides insulation to the gel packet 1 and the eyewear frame leg 3.

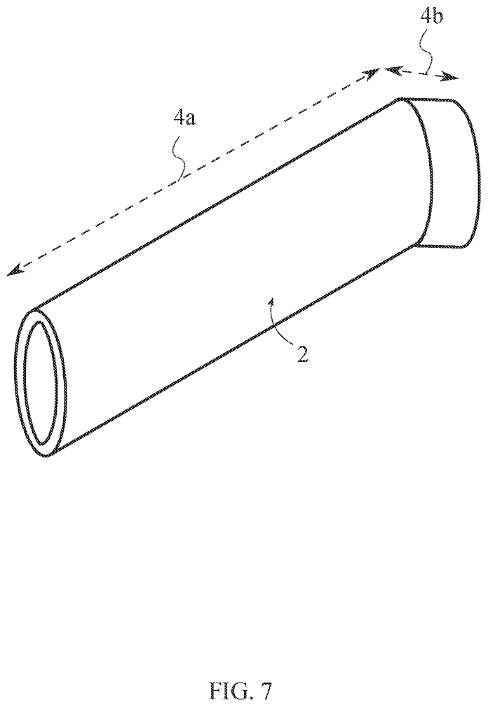

A more detailed description of the present invention follows. As seen in FIG. 7, the sleeve body 4 comprises a first section 4a and a second section 4b. Preferably, the first section 4a is terminally connected to the second section 4b, and the second section 4b is angularly offset from the first section 4a. Further, the first section 4a is longer than the second section 4b. This shape of the sleeve body 4 is provided so that the eyewear frame leg 3 can fit into the storage cavity 5 in a comfortable manner without affecting the natural shape of the eyewear. To that end, the eyewear frame leg 3 comprises a body section 3a and a tail section 3b. Preferably, the body section 3a is the longer horizontal segment of the eyewear frame that comes in contact with the temples of the user. Further, the tail section 3b is the smaller slanted section of the eyewear frame that goes behind the user's ears. Thus, when in use, the tail section is positioned within the second section 4b of the sleeve body 4, and the body section 3a is positioned within the first section 4a of the sleeve body 4. However, it should be noted that, the insulating sleeve 2 and the gel packet 1 may be made in any shape to fit eyewear frames of varying shapes and sizes. Thus, the present invention provides a versatile and easy to use cooling aid for using with eye wears.

Although the invention has been explained in relation to its preferred embodiment, it is to be understood that many other possible modifications and variations can be made without departing from the spirit and scope of the invention as hereinafter claimed.

What is claimed is:

1. A cold therapy device comprising:
a gel packet;
an insulating sleeve;
an eyewear frame leg;
the insulating sleeve comprising sleeve body, a storage cavity, a first end, and a second end;
the first end being positioned opposite the second end along a length of the insulating sleeve;
the storage cavity traversing through the sleeve body;
the storage cavity extending between the first end and the second end;
the gel packet being removably positioned within the storage cavity;
the insulating sleeve concealing the gel packet within the storage cavity; and
the eyewear frame leg being threaded through the insulating sleeve.

2. The cold therapy device of claim 1, wherein the gel packet within the insulating sleeve is pressed against the eyewear frame leg.

3. The cold therapy device of claim 1, wherein the gel packet is pre-cooled before being positioned within the insulating sleeve.

4. The cold therapy device of claim 1, wherein the insulating sleeve provides insulation to the gel packet and the eyewear frame leg.

5. The cold therapy device of claim 1, the sleeve body comprising:
a first section and a second section;
the first section being terminally connected to the second section; and
the second section being angularly offset from the first section.

6. The cold therapy device of claim 5, the eyewear frame leg comprising:
a body section and a tail section;
the tail section being positioned within the second section of the insulating sleeve; and
the body section being positioned within the first section of the insulating sleeve.

7. The cold therapy device of claim 5, wherein the first section is longer than the second section.

8. The cold therapy device of claim 1, wherein the storage cavity delineates a first hole adjacent to the first end and a second hole adjacent to the second end.

9. The cold therapy device of claim 1, the gel pack comprising:
a central cavity;
a plurality of sealed edges;
the central cavity being partially filled with gel; and
the sealed edges being perimetrically positioned around the central cavity.

10. The cold therapy device of claim 1, wherein the insulating sleeve being flexible.

11. The cold therapy device of claim 1, wherein at least one of the first end and the second end are sealable.

12. A cold therapy device comprising:
a gel packet;
an insulating sleeve;
an eyewear frame leg;
the insulating sleeve comprising sleeve body, a storage cavity, a first end, and a second end;
the first end being positioned opposite the second end along a length of the insulating sleeve;
the storage cavity traversing through the sleeve body;
the storage cavity extending between the first end and the second end;
the gel packet being removably positioned within the storage cavity, wherein the gel packet within the insulating sleeve is pressed against the eyewear frame leg;
the insulating sleeve concealing the gel packet within the storage cavity; and
the eyewear frame leg being threaded through the insulating sleeve, wherein the insulating sleeve provides insulation to the gel packet and the eyewear frame leg.

13. The cold therapy device of claim 12, the sleeve body comprising:
a first section and a second section;
the first section being terminally connected to the second section; and
the second section being angularly offset from the first section.

14. The cold therapy device of claim 13, the eyewear frame leg comprising:
a body section and a tail section;
the tail section being positioned within the second section of the insulating sleeve;
the body section being positioned within the first section of the insulating sleeve.

15. The cold therapy device of claim 13, wherein the first section is longer than the second section.

16. The cold therapy device of claim 12, wherein the storage cavity delineates a first hole adjacent to the first end and a second hole adjacent to the second end.

17. The cold therapy device of claim 12, the gel packet comprising:
a central cavity;
a plurality of sealed edges;
the central cavity being partially filled with gel; and
the sealed edges being perimetrically positioned around the central cavity.

18. The cold therapy device of claim 12, wherein the insulating sleeve being flexible.

19. The cold therapy device of claim 12, wherein at least one of the first end and the second end are sealable.

20. A cold therapy device comprising:
a gel packet;
an insulating sleeve;
an eyewear frame leg;
the insulating sleeve comprising sleeve body, a storage cavity, a first end, and a second end;
the sleeve body comprising a first section and a second section;
the first end being positioned opposite the second end along a length of the insulating sleeve;
the storage cavity traversing through the sleeve body;
the storage cavity extending between the first end and the second end;
the first section being terminally connected to the second section;
the second section being angularly offset from the first section
the gel packet being removably positioned within the storage cavity;
the insulating sleeve concealing the gel packet within the storage cavity; and
the eyewear frame leg being threaded through the insulating sleeve.
